(19) United States
(12) Patent Application Publication
(10) Pub. No.: US 2006/0134179 A1
(43) Pub. Date: Jun. 22, 2006

(54) Health food product

(75) Inventors: Kinya Takagaki, Fukuoka (JP); Takeshi Mitsui, Fukuoka (JP)

Correspondence Address: AMIN & TUROCY, LLP, 1900 EAST 9TH STREET, NATIONAL CITY CENTER, 24TH FLOOR,, CLEVELAND, OH, 44114, US

(73) Assignee: Toyo Shinyaku Co., Ltd.

(21) Appl. No.: 11/020,689

(22) Filed: Dec. 22, 2004

(51) Int. Cl. A61K 31/7048; A61K 31/353; A61K 31/198; A61K 47/00

(52) U.S. Cl. 424/439; 514/27; 514/456; 514/562

(57) ABSTRACT

The present invention provides a health food product comprising (A) proanthocyanidins, (B) ascorbic acid or a derivative thereof, and (C) L-cysteine or a derivative thereof. Preferably, proanthocyanidins containing at least 20 wt % of OPCs are used. The health food product of the present invention makes the body itself healthy and also provides an excellent beautification (skin-beautifying) effect.

### BACKGROUND OF THE INVENTION

1. Field of the Invention

The present invention relates to a health food product comprising proanthocyanidins, ascorbic acid or a derivative thereof, and L-cysteine or a derivative thereof.

2. Description of the Related Art

Conventionally, a variety of types of cosmetics have been used for the purpose of beautifying the skin so that the skin looks beautiful. However, since the cosmetics are used only topically or used to cope with a problem, beautiful skin in the true sense cannot be achieved. Since the condition of the skin reflects the health condition of, for example, blood vessels, the stomach and intestines, and the tissue inside the skin, it is preferable to maintain a good health condition of the body so as to maintain the skin beautiful.

In order to achieve this, a variety of types of health food products have been employed. For example, Japanese Laid-Open Patent Publication No. 8-205835 describes foods comprising collagen powder and an extract from Chinese gutta percha. However, these foods only supply effective components to the skin, and do not make the body itself healthy so as to make the skin beautiful. Health food products that contain ascorbic acid and L-cysteine in order to prevent pigmentation of the skin are commercially available. However, since ascorbic acid is metabolized in 2 to 3 hours after ingestion, its effect does not continue, and a certain amount of ascorbic acid has to be taken constantly. L-Cysteine in the food products works synergistically with ascorbic acid to prevent pigmentation of the skin. Thus, when the ascorbic acid has been metabolized in the body, the cysteine cannot exert its effect sufficiently.

Therefore, there is a demand for a health food product that makes the body itself healthy and provides an excellent effect of beautifying the skin.

### SUMMARY OF THE INVENTION

The present invention provides a health food product comprising (A) a proanthocyanidin, (B) ascorbic acid or a derivative thereof, and (C) L-cysteine or a derivative thereof. This health food product can make the body healthy and provide an excellent beautification (i.e., skin-beautifying) effect.

In a preferred embodiment, the proanthocyanidin comprise at least 20 wt % of OPC (oligomeric proanthocyanidin).

According to the present invention, the beautification (skin-beautifying) effect can be achieved by ingestion of the health food product comprising (A) proanthocyanidins, (B) ascorbic acid or a derivative thereof, and (C) L-cysteine or a derivative thereof. In particular, the excellent beautification (skin-beautifying) effect can be achieved by using proanthocyanidins comprising at least 20 wt % of OPC.

### DESCRIPTION OF THE PREFERRED EMBODIMENT

Hereinafter, the health food product of the present invention will be described. It should be noted that the following description is not limiting the present invention, and it is apparent to those skilled in the art that various modifications can be made within the scope of the spirit of the present invention.

The health food product of the present invention comprises (A) proanthocyanidins, (B) ascorbic acid or a derivative thereof, and (C) L-cysteine or a derivative thereof. Hereinafter, these components will be described.

(A) Proanthocyanidins

In the present invention, proanthocyanidins refer to a group of compounds that are condensation products having flavan-3-ol and/or flavan-3,4-diol as a constituent unit and having a degree of polymerization of 2 or more.

Proanthocyanidins are known to have various activities, such as providing antioxidation effect.

For the proanthocyanidins contained in the health food product of the present invention, ground products or extracts obtained from food raw material such as the bark of pine, oak, bayberry, and the like; the fruit or seeds of grape, blueberry, strawberry, avocado, locust, cowberry, and the like; the hull of barley, wheat, soybean, black soybean, cacao, adzuki bean, conker; the inner skin of peanuts; and the leaves of ginkgo can be used. In particular, it is preferable to use a pine bark extract.

In this specification, among proanthocyanidins, condensation products having flavan-3-ol and/or flavan-3,4-diol as a constituent unit and having a degree of polymerization of 2 to 4 are referred to as oligomeric proanthocyanidins (OPCs). OPCs, which are one type of polyphenol, are potent antioxidants produced by plants, and contained concentratedly in portions of plant leaves, bark, or skin or seeds of fruits. More specifically, they are contained in the seeds of grape; the bark of pine, oak, bayberry, and the like; the hull of conker; the inner skin of peanuts; the leaves of ginkgo; the fruit of locust; and the fruit of cowberry, for example. Moreover, it is known that OPCs are also contained in cola nuts in West Africa; the roots of Rathania in Peru; and Japanese green tea. OPCs cannot be produced in the human body.

Among proanthocyanidins, OPCs are especially abundant in pine bark as described above, and thus, pine bark is preferably used as a raw material of the proanthocyanidins in the present invention. Hereinafter, a method for preparing proanthocyanidins will be described taking a pine bark extract that contains OPCs abundantly as an example.

As the pine bark extract, an extract from the bark of plant belonging to Pinales, such as French maritime pine (Pinus martima), Larix leptolepis, Pinus thunbergii, Pinus densiflora, Pinus parviflora, Pinus pentaphylla, Pinus koraiensis, Pinus pumila, Pinus luchuensis, utsukushimatsu (Pinus densiflora form. umbraculifera), Pinus palustris, Pinus bungeana, and Anneda in Quebec, Canada, can be preferably used. Among these, French maritime pine (Pinus martima) bark extract is preferable.

French maritime pine refers to maritime pines that grow in a part of the Atlantic coastal area in southern France. It is known that the bark of this French maritime pine contains proanthocyanidins, organic acids, and other bioactive substances, and proanthocyanidins from the flavonoid family, which are the main component of the French maritime pine bark, have a potent antioxidation effect of removing active oxygen.

The pine bark extract is obtained by extracting the bark of the above-described pines using water or an organic solvent. When water is used, warm water or hot water can be employed, and the water may contain a salt such as sodium chloride. As the organic solvent that can be employed for extraction, an organic solvent that is acceptable for production of foods or pharmaceuticals can be employed. Examples of such solvent include methanol, ethanol, 1-propanol, 2-propanol, 1-butanol, 2-butanol, acetone, hexane, cyclohexane, propylene glycol, aqueous ethanol, aqueous propylene glycol, methyl ethyl ketone, glycerin, methyl acetate, ethyl acetate, diethyl ether, dichloromethane, edible oils or fats, 1,1,1,2-tetrafluoroethane, and 1,1,2-trichloroethene. These water and the organic solvents may be used alone or in combination. In particular, hot water, aqueous ethanol, and aqueous propylene glycol are preferably used.

The method for extracting proanthocyanidins from pine bark is not particularly limited, and heat extraction or supercritical fluid extraction can be employed, for example.

Supercritical fluid extraction is a method for performing extraction using a supercritical fluid. A supercritical fluid is in a state that is above the liquid-vapor critical point in the phase diagram showing critical temperature and critical pressure. Examples of compounds that can be employed as a supercritical fluid include carbon dioxide, ethylene, propane, and nitrous oxide (laughter gas). Carbon dioxide is preferably used.

Supercritical fluid extraction includes an extraction step in which a target component is extracted with a supercritical fluid and a separation step in which the target component is separated from the supercritical fluid. In the separation step, any separation process can be employed, examples of which include a separation based on a change in pressure, a separation based on a change in temperature, and a separation using an adsorbent or absorbent.

Moreover, it is also possible to perform supercritical fluid extraction in which an entrainer is added. In this method, extraction is performed using an extracting fluid obtained by adding, for example, ethanol, propanol, n-hexane, acetone, toluene, or another aliphatic lower alcohol, aliphatic hydrocarbon, aromatic hydrocarbon, or ketone at about 2 to 20 W/V% to a supercritical fluid, so that the solubility of a target substance to be extracted, such as OPCs and catechins (described later), in the extracting fluid is dramatically increased or the selectivity of separation is enhanced. Thus, a pine bark extract is obtained efficiently.

Since supercritical fluid extraction can be performed at a relatively low temperature, it has the following advantages: it is applicable for extracting substances that deteriorate or decompose at high temperatures; the extracting fluid does not remain; and the extracting fluid can be recovered and recycled, so that a step of removing the extracting fluid and the like can be omitted, and thus, the process can be simplified.

Furthermore, methods other than those mentioned above can be employed for extraction from pine bark, and the examples of which include a batch method using liquid carbon dioxide, a reflux method using liquid carbon dioxide, a reflux method using supercritical carbon dioxide, and the like.

It is also possible to employ a combination of a plurality of extraction processes to perform extraction from pine bark. By combining a plurality of extraction processes, pine bark extracts with various components can be obtained.

The pine bark extract that is used for the health food product of the present invention is specifically prepared using the following method. However, this method is merely an example and the present invention is not limited to this method.

First, 1 kg of the bark of French maritime pine is immersed in 3 L of a saturated solution of sodium chloride, and extraction is performed for 30 minutes at 100° C. to obtain an extract liquid (extraction step). Then, the extract liquid is filtrated, and the resultant insoluble material is washed with 500 ml of a saturated solution of sodium chloride to obtain a washed liquid (washing step). The extract liquid and the washed liquid are combined to obtain a crude extract liquid of pine bark.

Next, 250 ml of ethyl acetate is added to this crude extract liquid, mixed, and separated to obtain an ethyl acetate layer. This process is repeated five times, and the obtained ethyl acetate layers are combined. The resultant ethyl acetate extract is added directly to 200 g of anhydrous sodium sulfate for dehydration. Then, this ethyl acetate extract is filtrated, and the filtrated extract is concentrated under a reduced pressure to a volume of ⅕ of the original filtrated extract. The concentrated ethyl acetate extract is poured into 2 L of chloroform and stirred, and the resultant precipitate is recovered by filtration. Subsequently, this precipitate is dissolved in 100 ml of ethyl acetate, and then the resultant solution is added to 1 L of chloroform to form a precipitate. This process is repeated twice, and thus, a washing process is accomplished. With this method, for example, about 5 g of pine bark extract containing at least 20 wt % of OPCs that have a degree of polymerization of 2 to 4 and at least 5 wt % of catechins can be obtained.

Extracts from the above-described raw material plants, in particular, pine bark extracts, which are typically used for proanthocyanidins (A) in the health food product of the present invention, contain proanthocyanidins that are condensation products having flavan-3-ol and/or flavan-3,4-diol as a constituent unit and having a degree of polymerization of 2 or more. Among these, extracts that contain a large amount of condensation products having a low degree of polymerization are preferably used. As such condensation products, condensation products having a degree of polymerization of 2 to 30 (dimer to 30-mer) are preferable, condensation products having a degree of polymerization of 2 to 10 (dimer to decamer) are more preferable, and condensation products having a degree of polymerization of 2 to 4 (dimer to tetramer; i.e., OPCs) are even more preferable.

Since OPCs are antioxidants as described above, they also provide an effect of reducing the possibility of adult diseases, such as cancer, cardiac diseases, and cerebral thrombosis, an effect of improving allergic diathesis, such as arthritis, atopic dermatitis, and pollenosis, an effect of inhibiting oxidation and degradation of collagen, and the like. Furthermore, OPCs allow vitamin C (ascorbic acid) to be used efficiently in the body, and enhance antioxidation ability in the body in cooperation with vitamin C (ascorbic acid).

Furthermore, it is known that in addition to the antioxidation effect, OPCs also provide, for example, an effect of inhibiting bacterial proliferation in the oral cavity to reduce plaque (dental plaque); an effect of recovering the elasticity of blood vessels; an effect of preventing lipoprotein in blood from being damaged by active oxygen, thereby preventing aggregation and adherence of the oxidized fats onto the inside wall of the vessel, thus preventing cholesterol from being aggregated and adhered onto the oxidized fats that have been adhered onto the inside wall of the vessel; an effect of regenerating vitamin E that has been degraded by active oxygen; and an effect of serving as an enhancer of vitamin E. Among these, in particular, by virtue of the effect of recovering the elasticity of blood vessels and the effect of preventing adhesion of cholesterol, circulation of blood in the body is promoted, and thus, the metabolism of the skin is activated, and the skin is maintained in good health.

In the present invention, proanthocyanidins containing at least 20 wt % of OPCs are preferably used. More preferably, the OPC content is at least 30 wt %. As such proanthocyanidins, a pine bark extract is preferably used.

When proanthocyanidins having a high OPC content are used, a better effect of beautifying the skin can be achieved than in the case where proanthocyanidins having a high degree of polymerization (i.e., having a low OPC content) are used.

Moreover, proanthocyanidin contents derived from raw material plants, in particular, plant extracts contain catechins as well as OPCs. The term “Catechins” is a general term referring to polyhydroxyflavan-3-ols. As the catechins, for example, (+)-catechin, (−)-epicatechin, (+)-gallocatechin, (−)-epigallocatechin, epigallocatechin gallate, and epicatechin gallate are known. Gallocatechin, afzelechin, and 3-galloyl derivatives of (+)-catechin or gallocatechin are isolated from natural products, in addition to (+)-catechin that is called catechin in a narrow sense. Catechins are known to have a cancer inhibiting ability, an arteriosclerosis preventing ability, a lipid metabolism disorder inhibiting ability, a blood pressure elevation inhibiting ability, a turombosis preventing ability, an antiallergic ability, an antiviral ability, an antibacterial ability, a dental caries preventing ability, a halitosis preventing ability, an intestinal flora normalization ability, an active oxygen or free radical eliminating ability, an antioxidation effect, and the like. Moreover, catechins are known to have an antidiabetic ability that inhibits an elevation of blood glucose. Furthermore, catechins have the property of both increasing the solubility in water and being activated in the presence of OPCs.

It is preferable that catechins are contained in the above-described raw material plant extracts in a ratio of 5 wt % or more. Alternatively, it is also preferable that a formulation is prepared so that it contains a raw material plant extract containing at least 20 wt % of OPCs, and furthermore, contains catechins in a ratio of 5 wt % or more. For example, when the catechin content in a pine bark extract is less than 5 wt %, it is possible to add catechins so that the catechin content becomes at least 5 wt %. It is most preferable to use a pine bark extract containing at least 5 wt % of catechins and at least 20 wt % of OPCs.

(B) Ascorbic Acid and Derivatives Thereof

As ascorbic acid or a derivative thereof to be contained in the health food product of the present invention, ascorbic acid and derivatives thereof that are used as food additives, such as sodium ascorbate, magnesium ascorbate, and ascorbyl glycoside, can be used. Natural materials that contain ascorbic acid abundantly (e.g., natural materials derived from fruits such as lemon, orange, and acerola, or natural materials derived from vegetables such as broccoli, Brussels sprouts, pimento, Brassica campestris, and cauliflower) also can be used as the ascorbic acid in the present invention.

Ascorbic acid is contained in order to inhibit biosynthesis of melanin, which may cause a darkening of the skin and the like, by an antioxidation effect or an ability of inhibiting tyrosinase activity. Furthermore, ascorbic acid is known to have an ability of promoting synthesis of collagen, which is a structural protein of the skin, an ability of reducing stresses (in particular, oxidative stress), and an ability of increasing immunopotency.

The proanthocyanidins (A) and ascorbic acid or a derivative thereof (B) are contained in the health food product of the present invention at a weight ratio of preferably in the range of 1:0.1 to 1:50 and more preferably 1:0.2 to 1:20. It should be noted that the amount of ascorbic acid may be larger than the ratios described above.

Ascorbic acid or a derivative thereof (B) is contained in the health food product of the present invention in a ratio of at least 20 parts by weight with respect to a total amount of 100 parts by weight of proanthocyanieins (A) and L-cysteine or a derivative thereof (C).

(C) L-Cysteine and Derivatives Thereof

Another essential component of the health food product of the present invention is L-cysteine or a derivative thereof. L-cysteine and derivatives thereof, which are amino acid having a SH group, act synergistically with ascorbic acid to provide a tyrosinase inhibition activity and an activity of making melanin pigment colorless. In addition to these, they are known to provide an ability of regulating metabolism of the epidermis, an ability of improving liver function, an ability of promoting detoxication, and the like. Examples of the derivatives of L-cysteine include, but are not limited to, L-cystine (i.e., dimer of L-cysteine) and N-acetylcysteine.

The L-cysteine or a derivative thereof used for the health food product of the present invention may be a chemical compound or a material derived from a natural product. Examples of the material derived from a natural product include wheat protein, soybean protein, powdered skim milk, extracts such as yeast extract, and purified products thereof Furthermore, a natural product containing these substances may be contained without any treatment.

The weight ratio of proanthocyanidins (A) and L-cysteine or a derivative thereof (C) is preferably in the range of 1:0.2 to 1:40 and more preferably 1:0.5 to 1:25. The ascorbic acid or a derivative thereof (B) is contained in the health food product of the present invention preferably in the range of 0.1 to 50 parts by weight, and more preferably in the range of 0.2 to 20 parts by weight based on 1 part by weight of the proanthocyanidins (A). The L-cysteine or a derivative thereof (C) is contained in the health food product of the present invention preferably in the range of 0.2 to 40 parts by weight, and more preferably in the range of 0.5 to 25 parts by weight based on 1 part by weight of the proanthocyanidins (A).

The health food product of the present invention contains the above-described (A) to (C), and also contains a variety of types of additives that are commonly used for foods, if necessary. Examples of such additives include excipients, extenders, binders, thickners, emulsifiers, coloring agents, flavors, and food additives. For example, the health food product of the present invention may be produced in the form of, for example, tablets or pills by adding an excipient and the like to a pine bark extract containing proanthocyanidins abundantly, ascorbic acid, and L-cysteine, or it may be produced in the form of powder or in other forms without being shaped.

It is also possible to made into the forms of capsules such as hard capsules and soft capsules, powder, granule, tea bags, candy, liquid, and paste.

Furthermore, nutritions, such as royal jelly, vitamins, proteins, calcium substances such as eggshell calcium, chitosan, lecithin, chlorella powder, Angelica keiskei powder, and molokheiya powder, also can be added. It is also possible to add stevia powder, ground green tea powder, lemon powder, honey, maltitol, lactose, sugar solutions, seasonings, and the like so as to control taste.

Regarding the method for ingesting the health food product of the present invention, there is no particular limitation. According to the form of the health food product of the present invention or according to the preference, the health food product may be eaten or drunk as it is, or may be dissolved in water, hot water, milk, or the like and drunk. Alternatively, a liquid containing the components of the health food product obtained by percolation may be drunk.

Although there is no limitation regarding the daily intake amount of the health food product of the present invention, it is preferable that the amount of proanthocyanidins (A) is 20 to 1000 mg, the amount of ascorbic acid or a derivative thereof (B) is 100 mg to 1000 mg, and the amount of L-cysteine or a derivative thereof (C) is 10 to 500 mg.

It seems that the health food product of the present invention not only provides a whitening effect, which is achieved by the antioxidation effect and the tyrosinase inhibition ability of its components, but also provides an excellent effect of beautifying the skin because various bioactivities of the components (A) to (C) cooperate to improve the health condition of the body and activate the metabolism so as to maintain a healthy skin condition. Furthermore, it also provides an excellent beautification effect because it makes the skin beautiful.

### EXAMPLES

Hereinafter, the present invention will be described by way of examples. However, the present invention is not limited to these examples.

### Example 1

First, tablets (about 220 mg per tablet) were produced using (A) an ethanol extract of pine bark (trade name: Flavangenol, produced by TOYO SHINYAKU Co., Ltd.) containing 40 wt % of proanthocyanidins (OPC content: 20 wt % in the extract) and 5 wt % of catechins, (B) ascorbic acid (Maruzen Pharmaceuticals Co., Ltd.), (C) L-cystine (Nippon Bulk Yakuhin Co., Ltd.), crystalline cellulose, sucrose ester, silicon dioxide, and eggshell calcium in a ratio shown in Table 1 below to obtain a food.

Then, twenty experimental subjects (women between the ages of 23 and 45) who were afflicted with blotches and freckles ingested 12 tablets of this food daily for four weeks. After the ingestion, the subjects performed self-assessment of the beautification (skin-beautifying) effect (evaluated regarding vitality, luster, blotches and freckles, and darkening of their skin) using the criteria listed below. The subjects were divided into groups randomly except that the number of subjects of similar age was equal among the groups:

| | | |
|---|---|---|
| Significantly improved: | 2 | points |
| Improved: | 1 | point |
| Unchanged: | 0 | points |

Table 2 shows the results. The results in Table 2 indicate the averages of the marks given by the twenty subjects.

### Example 2

Tablets (food) were produced by the use of the components shown in Table 1 below in the same manner as in Example 1 except that an ethanol extract of pine bark containing 40 wt % of proanthocyanidins (OPC content: 10 wt % in the extract) and 5 wt % of catechins was used in place of (A) in Example 1, and the beautification (skin-beautifying) effect was assessed. Table 2 also shows the results.

### Comparative Examples 1 to 3

Tablets (foods) were produced by the use of the components shown in Table 1 below that include only one or two of (A) to (C), and the beautification (skin-beautifying) effect was assessed in the same manner as in Example 1. Table 2 also shows the results.

TABLE 1

| | | | | Com. | Com. | Com. |
|---|---|---|---|---|---|---|
| | Components | Ex. 1 | Ex. 2 | Ex. 1 | Ex. 2 | Ex. 3 |
| (A) | Pine bark extract | 10 | — | 20 | 20 | — |
| | (OPC content: 20 wt %) | | | | | |
| | Pine bark extract | — | 10 | — | — | — |
| | (OPC content: 10 wt %) | | | | | |
| (B) | Ascorbic acid | 5 | 5 | — | 5 | 5 |
| (C) | L-Cystine | 6 | 6 | — | — | 12 |
| Additives | Crystalline cellulose | 10 | 10 | 10 | 10 | 10 |
| | Sucrose ester | 5 | 5 | 5 | 5 | 5 |
| | Silicon dioxide | 2 | 2 | 2 | 2 | 2 |
| | Eggshell calcium | 62 | 62 | 63 | 58 | 66 |

The unit of the values: parts by weight.

TABLE 2

| | | | Com. | Com. | Com. |
|---|---|---|---|---|---|
| Response items | Ex. 1 | Ex. 2 | Ex. 1 | Ex. 2 | Ex. 3 |
| Vitality of skin | 1.2 | 1 | 0.4 | 0.5 | 0.65 |
| Luster of skin | 1.25 | 0.9 | 0.35 | 0.4 | 0.5 |
| Blotches and freckles of skin | 1.55 | 1.15 | 0.45 | 0.6 | 0.9 |
| Darkening of skin | 1.5 | 1.2 | 0.2 | 0.65 | 0.85 |

The values indicate the average marks.

Referring to the results shown in Table 2, it can be recognized that the foods of the examples provide a higher beautification (skin-beautifying) effect in all of the items. Furthermore, it can be recognized that the food of Example 1 provides a higher beautification (skin-beautifying) effect than the food of Example 2 does. Moreover, the subjects who ingested the foods of the examples made more remarks that stiffness in the shoulders and lower back pain disappeared or tiredness disappeared than the subjects who ingested the foods of the comparative examples did.

The invention may be embodied in other forms without departing from the spirit or essential characteristics thereof. The embodiments disclosed in this specification are to be considered in all respects as illustrative and not limiting. The scope of the invention is indicated by the appended claims rather than by the foregoing description, and all changes which come within the meaning and range of equivalency of the claims are intended to be embraced therein.

1. A health food product, comprising (A) a proanthocyanidin, (B) ascorbic acid or a derivative thereof, and (C) L-cysteine or a derivative thereof.

2. The health food product of claim 1, wherein the proanthocyanidin comprises at least 20 wt % of OPC (oligomeric proanthocyanidin).
